(19) United States
(12) Patent Application Publication
(10) Pub. No.: US 2019/0308028 A1
(43) Pub. Date: Oct. 10, 2019

(54) SUPPORT BRACE ACCOMMODATING A MEDICAL DEVICE

(71) Applicant: Lee H. White, Springboro, OH (US)

(72) Inventor: Lee H. White, Springboro, OH (US)

(21) Appl. No.: 16/379,940

(22) Filed: Apr. 10, 2019

Related U.S. Application Data
(60) Provisional application No. 62/655,597, filed on Apr. 10, 2018.

(51) Int. Cl. A61N 2/00; A61F 5/01; A61N 2/02

(52) U.S. Cl. CPC A61N 2/002; A61N 2/02; A61N 2/008; A61F 5/0109

(57) ABSTRACT

A support brace comprising adjustable features and an attachment device. The attachment device, such as a pocket, may be configured to receive one or more pain relief or rehabilitation assistance devices. The pain relief or rehabilitation assistance devices may be an electromagnetic pulse therapy device.

### CROSS-REFERENCE TO RELATED APPLICATION

This application claims the benefit of U.S. Provisional Application No. 62/655,597 filed Apr. 10, 2018, the disclosures of which are hereby incorporated by reference as if fully restated herein.

### TECHNICAL FIELD

Exemplary embodiments of the present invention relate generally to support braces.

### BACKGROUND AND SUMMARY OF THE INVENTION

Support braces have long been used to enhance user stability. Such support braces are braces typically configured to be placed on an individual body part. For example, without limitation, such support braces may be configured to be placed on the user's knee, elbow, wrist, back, neck, or ankle. Some exemplary support braces may comprise a wrap configured to be wrapped around a joint. Other support braces may comprise a piece of material configured to held to a particular body part, such as by use of an adhesive. Some support braces are configured to be adjustable in various ways. Such adjustability may be achieved, for example without limitation, by the use of stretching material, straps, fasteners, adhesives, cut outs, or the like.

Support braces are typically used to assist a user with an otherwise unstable joint or as a relief from pain or swelling, particularly when rehabilitating from an injury. Various medical devices have been used separately from such support braces to help alleviate pain, assist in rehabilitation, or otherwise increase healing. However, the support brace must generally be removed to use the separate pain relief or rehabilitation assistance devices. This is particularly problematic when a user is mobile or exercising, which is often when the most pain is incurred or assistance is most needed. Therefore, what is needed is a support brace configured to accommodate one or more pain relief or rehabilitation assistance devices.

The present invention is a support brace configured to accommodate one or more pain relief or rehabilitation assistance devices. Exemplary embodiments may comprise an adjustable support brace and an attachment device configured to receive one or more pain relief or rehabilitation assistance devices. In some exemplary embodiments, the support brace is a sleeve. In one exemplary embodiment, the attachment device is a pocket. In a further exemplary embodiment, the pain relief or rehabilitation assistance device is an electromagnetic pulse therapy device. The electromagnetic pulse therapy device may be, but is not limited to, the ActiPatch® device manufactured by BioElectroincs (http://www.bielcorp.com/products/actipatch/).

Further features and advantages of the systems and methods disclosed herein, as well as the structure and operation of various aspects of the present disclosure, are described in detail below with reference to the accompanying figures.

### BRIEF DESCRIPTION OF THE DRAWINGS

In addition to the features mentioned above, other aspects of the present invention will be readily apparent from the following descriptions of the drawings and exemplary embodiments, wherein like reference numerals across the several views refer to identical or equivalent features, and wherein:

FIG. 1 is a front perspective view of an exemplary support brace;

FIG. 2 is an interior view of the exemplary support brace of FIG. 1; and

FIG. 3 is an interview view of another exemplary embodiment of the support brace of FIG. 1.

### DETAILED DESCRIPTION OF EXEMPLARY EMBODIMENT(S)

Various embodiments of the present invention will now be described in detail with reference to the accompanying drawings. In the following description, specific details such as detailed configuration and components are merely provided to assist the overall understanding of these embodiments of the present invention. Therefore, it should be apparent to those skilled in the art that various changes and modifications of the embodiments described herein can be made without departing from the scope and spirit of the present invention. In addition, descriptions of well-known functions and constructions are omitted for clarity and conciseness.

Embodiments of the invention are described herein with reference to illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

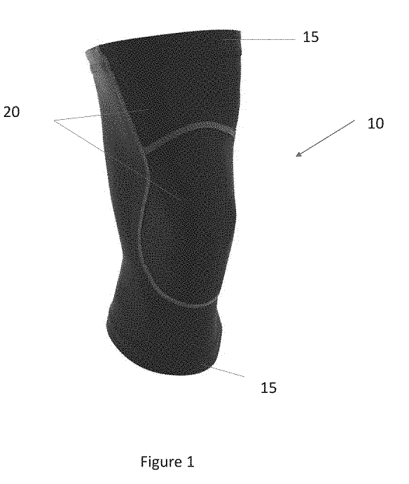

FIG. 1 illustrates an exemplary support brace 10. The support brace 10 may be configured to be placed on a user's body to increase stability of, and provide compression to, one or more body parts. The support brace 10 may comprise one or more compression or stability device, such as but not limited to, compression materials, rods, stiffening members, braces, or the like. In exemplary embodiments, the support brace 10 may be adjustable or sized to fit various size users. For example, without limitation, the support brace 10 may be a wrap configured to be wrapped around a joint. As a further example, again without limitation, the support brace 10 may be a section of material configured to be secured to a body part, such as by use of an adhesive. In still another example, again without limitation, the support brace 10 may be configured to be secured to one or more body parts such as but not limited to, a user's knee, elbow, wrist, back, neck, or ankle. In some exemplary embodiments, the support brace is a sleeve.

The support brace 10 may comprise various attachment and adjustment devices configured to secure the support brace 10 to the user's respective body part. Such attachment and adjustment devices may include, without limitation, the use of stretchable sections 20, stretchable bands 15, straps, fasteners, buckles, laces, adhesives, cut outs, or the like. The support brace 10, including but not limited to the stretchable sections 20 and the stretchable bands 15, may be comprised of a two-, three-, or four-way stretchable material such as but not limited to, latex, elastic, neoprene, or the like. All or some of the support brace 10, including but not limited to the stretchable sections 20 and the stretchable bands 15, may be coated with an anti-slip material to prevent the support brace 10 from slipping during wear. The support brace 10 may further comprise padded sections. The support brace 10 may further comprise one or more supporting elements such as, but not limited to, a stiffening member, a rod, straps, compression devices, and the like.

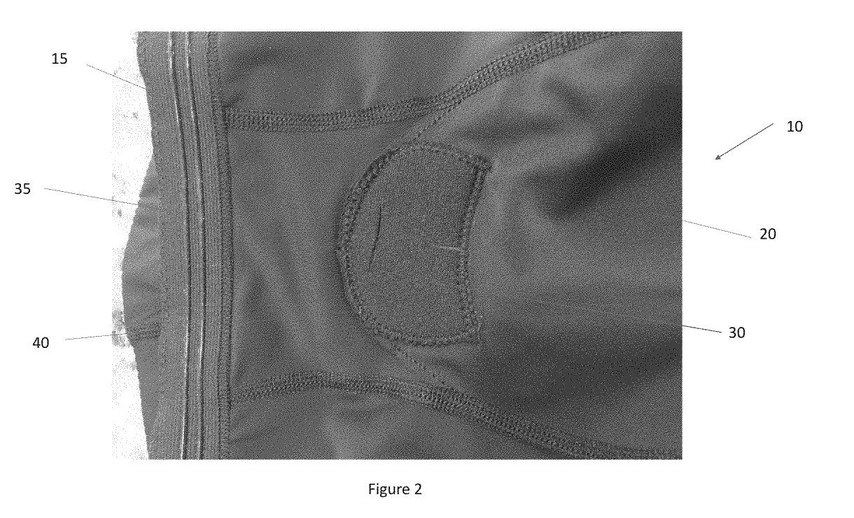

FIG. 2 illustrates an interior view of an exemplary support brace 10. The support braces 10 may comprise an attachment device 30 configured to accommodate one or more pain relief or rehabilitation assistance devices 45. The attachment device 30 may secure the one or more pain relief or rehabilitation assistance devices 45 to the support brace 10 around a particular location on the user's body. For example, without limitation, the attachment device 30 may be a pocket. The use of one or more pockets is contemplated. The pocket may be sewn, welded, adhered, fastened, or otherwise bonded to the support brace 10. In exemplary embodiments, the pocket material is sewn so such that the top to bottom will be in the weft direction, thereby allowing more longitudinal stretch. In other exemplary embodiments, the remaining portions of the pocket and support brace 10 may be sewn in the warp direction, thereby allowing more latitudinal stretch. Regardless, the pocket may comprise one or more slits 35 or other apertures located on or around the pocket and configured to permit the one or more pain relief or rehabilitation assistance devices 45 to be selectively inserted into and removed from the pocket. The pocket may further comprise an elastomeric pad 40 configured to provide a cushion between the pain relief or rehabilitation assistance device 45 located in the pocket 30 and the user's skin. In exemplary embodiments, the pain relief or rehabilitation assistance device 45 may be an electromagnetic pulse therapy device, such as but is not limited to, the ActiPatch® device manufactured by BioElectroincs (http://www.bielcorp.com/products/actipatch/).

In an exemplary embodiment, the support brace 10 is a knee brace configured to be worn about the user's knee. The support brace 10 may be comprised of one or more stretchable sections 20 and comprise one or more stretchable bands 15 configured to secure the support brace 10 to the user's knee. The support brace 10 may further comprise an attachment device 30 in the form of a pocket, which may be sized and configured to accommodate a pain relief or rehabilitation assistance device 45 including the Acti Patch® device. For example, without limitation, the attachment device 30 may be located so as to provide electromagnetic therapy to the upper center of the knee so as to relieve common knee pain.

Any number, location, size, shape, orientations, or type of attachment devices 30 are contemplated.

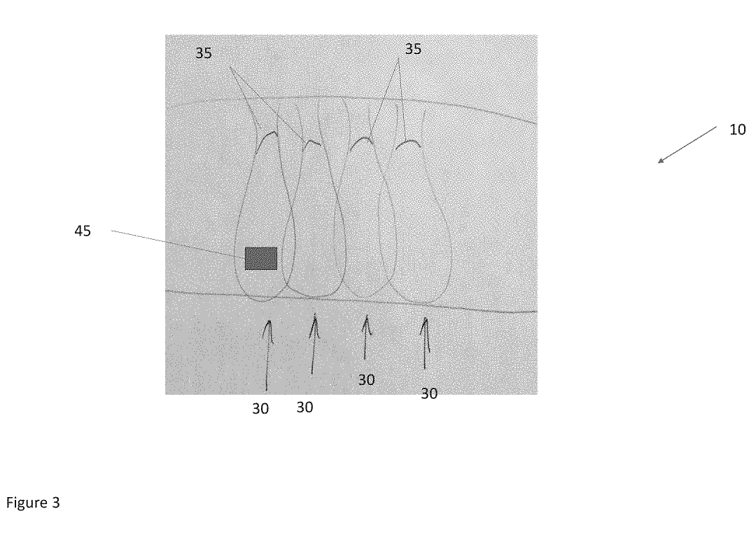

As illustrated in FIG. 3 multiple attachment devices 30, such as but not limited to pockets, may be located around an area for treatment. In this way, for example, without limitation, the pain relief or rehabilitation assistance device 45 may be moved from one attachment device 30 to another to best locate the pain relief or rehabilitation assistance device 45. As another example, without limitation, multiple pain relief or rehabilitation assistance device 45 may be used. The attachment devices 30 may be aligned in a substantially linear fashion along the support brace 10, though such is not required. The attachment devices 30 may be positioned along an area of common injury for the body part. For example, without limitation, the attachment device 30 may be located within the support brace 10 to align with the anterior cruciate ligament (ACL), the posterior cruciate ligament (PCL), the medial collateral ligament (MLC), the lateral collateral ligament (LCL) the lateral meniscus, or the medial meniscus of the knee.

In one exemplary embodiment, without limitation, multiple attachment device 30 may be located along a knee brace to permit positioning of the pain relief or rehabilitation assistance device 45 where the user is experiencing pain. In another exemplary embodiment, without limitation, the support brace 10 may be configured to be placed along the user's back and the attachment device 30 may be located in spaced intervals vertically to correspond with locations along the user's spinal column such that the pain relief or rehabilitation assistance device 45 may be positioned at various locations along the user's spine.

Any embodiment of the present invention may include any of the optional or preferred features of the other embodiments of the present invention. The exemplary embodiments herein disclosed are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, those skilled in the art will realize that many variations and modifications may be made to the described invention. Many of those variations and modifications will provide the same result and fall within the spirit of the claimed invention. It is the intention, therefore, to limit the invention only as indicated by the scope of the claims.

What is claimed is:

1. A support brace for a body part of a user comprising:
one or stretchable sections configured to accommodate the body part; and
one or more attachment devices, each configured to receive a pain relief or rehabilitation assistance device.

2. The support brace of claim 1 wherein:
the support brace is in the form of a sleeve.

3. The support brace of claim 2 wherein:
the body part is the knee; and
the sleeve is configured to surround the knee of the user.

4. The support brace of claim 1 wherein:
each of the one or more attachment devices is a pocket; and
each pocket is sewn to an interior surface of the support brace.

5. The support brace of claim 4 wherein:
the pain relief or rehabilitation assistance device is an electromagnetic pulse therapy device.

6. The support brace of claim 5 wherein:
each of the pockets are sewn such that the top to bottom is in the weft direction.

7. The support brace of claim 5 wherein:
each of the pockets are sewn such that the top to bottom is in the warp direction.

8. The support brace of claim 5 further comprising:
a slit located in each of the pockets sized to receive the electromagnetic pulse therapy device.

9. The support brace of claim 8 wherein:
the one or more attachment devices comprises at least four pockets;
each of the at least four pockets extend along the support brace in a linear fashion; and
each of the at least four pockets are positioned to align with one or more areas of common injury or pain for the body part.

10. The support brace of claim 1 further comprising:
one or more devices for securing the support brace to the body part.

11. The support brace of claim 11 wherein:
each of the one or more devices for securing the support brace to the body part are stretchable bands.

12. A support brace for a body part of a user comprising:
one or stretchable sections configured to accommodate the body part;
multiple pockets sewn to an interior surface of the support brace; and
a series of slits, each slit being located in one of the multiple pockets;
wherein each pocket is configured to receive an electromagnetic pulse therapy device.

13. The support brace of claim 12 wherein:
the stretchable sections are comprised of a four-way stretch material.

14. The support brace of claim 12 wherein:
the multiple pockets comprise at least three pockets;
each of the at least three pockets extend along the interior surface in a linear fashion; and
each of the at least three pockets are positioned to align with one or more areas of common injury or pain for the body part.

15. The support brace of claim 14 wherein:
the body part is the knee; and
the pockets are positioned to be aligned with the anterior cruciate ligament (ACL).

16. The support brace of claim 14 wherein:
the body part is the knee; and
the pockets are positioned to be aligned with the posterior cruciate ligament (PCL).

17. The support brace of claim 14 wherein:
the body part is the knee; and
the pockets are positioned to be aligned with the medial collateral ligament (MLC).

18. The support brace of claim 14 wherein:
the body part is the knee; and
the pockets are positioned to be aligned with the lateral collateral ligament (LCL).

19. The support brace of claim 14 wherein:
the body part is the knee; and
the pockets are positioned to be aligned with the lateral meniscus or the medial meniscus.

20. The support brace for a knee of a user comprising:
a sleeve comprising:
one or stretchable sections configured to accommodate the knee of the user, wherein each of the stretchable sections are comprised of a four-way stretch material;
an upper stretchable band configured to be secured to the upper leg; and
a lower stretchable band configured to be secured to the lower leg;
a series of pockets sewn along an interior surface of the sleeve in a substantially linear fashion, wherein each of the series of pockets is configured to receive an electromagnetic pulse therapy device; and
a slit located in each of the pockets, wherein the slit is configured to permit the electromagnetic pulse therapy device to be inserted into the pocket.
